(19) United States
(12) Patent Application Publication
(10) Pub. No.: US 2024/0287135 A1
(43) Pub. Date: Aug. 29, 2024

(54) ANTIMICROBIAL PEPTIDE OR PEPTIDE DERIVATIVE, SUBSTITUTE, AND COMPOSITION THEREOF, AND PREPARATION METHOD AND APPLICATION OF COMPOSITION

(71) Applicant: QC BIO-TECHNOLOGY (SHENZHEN) CO., LTD., Shenzhen, Guangdong (CN)

(72) Inventors: Tong WEN, Taizhou, Jiangsu (CN); Wen WEN, Taizhou, Jiangsu (CN)

(21) Appl. No.: 18/022,739

(22) PCT Filed: Sep. 3, 2021
(86) PCT No.: PCT/CN2021/116530

§ 371 Date: Feb. 22, 2023

(51) Int. Cl. C07K 7/08; A61K 38/00; C07K 14/00

(52) U.S. Cl. CPC C07K 7/08; C07K 14/001; A61K 38/00

(57) ABSTRACT

An antimicrobial peptide (AMP) or peptide derivative, a substitute for the AMP or peptide derivative, an AMP or peptide derivative composition, and a preparation method and application of the AMP or peptide derivative composition. The AMP or peptide derivative includes at least one selected from the group consisting of amino acid sequences I and II:

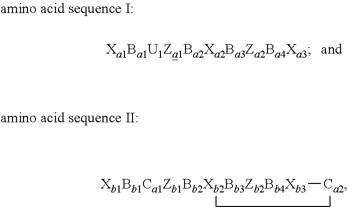

where Xₐ₁, Bₐ₁, U₁, Zₐ₁, Bₐ₂, Xₐ₂, Bₐ₃, Zₐ₂, Bₐ₄, Xₐ₃, X_{b1}, B_{b1}, Cₐ₁, Z_{b1}, B_{b2}, X_{b2}, B_{b3}, Z_{b2}, B_{b4}, X_{b3}, and Cₐ₂ each are independently selected from the group consisting of natural amino acids (NAAs) and unnatural amino acids (UAAs). The AMP or peptide derivative provided by the present application can combine with a lipid structure of a cell wall/membrane to damage its physical and chemical properties, thereby destroying the microbial wall/membrane to kill microorganisms and tumor cells.

### CROSS-REFERENCE TO RELATED APPLICATIONS

This application is a U.S. national phase of International Patent Application No. PCT/CN2021/116530 with an international filing date of Sep. 3, 2021, designating the U.S., now pending, the contents of which are incorporated herein by reference.

### INCORPORATION-BY-REFERENCE OF SEQUENCE LISTING

The Sequence listing, named “SEQUENCE LISTING”, created on Feb. 22, 2023 with 16,384 size in bytes is incorporated by reference in this application.

### TECHNICAL FIELD

The present application belongs to the technical field of peptides or peptide derivatives, and in particular, to an antimicrobial peptide (AMP) or peptide derivative, a substitute for the AMP or peptide derivative, an AMP or peptide derivative composition, and a preparation method and application of the AMP or peptide derivative composition.

### BACKGROUND

In nature, AMPs are widely present in organisms such as bacteria, viruses, fungi, insects, amphibians, animals, and plants, and AMPs play a very significant role in killing bacteria, fungi, parasites, and viruses. With the large consumption of antibiotics, microorganisms have developed antibiotic resistance, which promotes and drives the large-scale research and development of AMPs by research and development personnel. Since human endogenous AMPs such as defensins are discovered and approved by the Food and Drug Administration (FDA) as drugs for anti-infection, anti-inflammation, and other injury treatments, it has been further discovered that AMPs can stimulate innate and acquired immune effects in a body, indicating the potential therapeutic advantages of AMPs for some diseases. In addition to medical drugs, AMPs are widely used in food preservation, poultry husbandry, aquatic fishery, daily medical health care, hygiene and beauty, cleaning and disinfection, and agricultural disease and pest control.

For example, acne is caused by the overgrowth of the gram-positive bacterium Propionibacterium acnes (P. acnes) (a microbial flora on normal human skin) in pilosebaceous follicles. Acne is one of the most common diseases affecting human skin, and approximately one million Chinese suffer from acne. Acne caused by P. acnes has different manifestations, such as pimples, papules, tuberculosis, cysts, and inflammation of pilosebaceous follicles. Traditionally, acne caused by P. acnes is mostly treated through antibiotic administration, such as oral administration of oxytetracycline (OTC) and external administration of erythromycin or clindamycin, but such treatment methods quickly leads to antibiotic resistance. In addition, acne caused by P. acnes is clinically treated with benzoyl peroxide and 5% dapsone gel, but these two drugs exhibit low efficacy at a low concentration and have great toxic and side effects at a high concentration.

For example, Pseudomonas aeruginosa (P. aeruginosa) is a gram-negative bacterium invading the human and animal bodies, which can infect the respiratory system, urinary system, gastrointestinal system, central nervous system (CNS), blood system, cardiac system, and skeletal system of an organism. When infecting the eyes and ears, P. aeruginosa often causes bacterial keratitis, scleral abscess, and endophthalmitis in adults, ophthalmia neonatorum, and pediatric otitis media. In particular, P. aeruginosa increases the bacterial keratitis prevalence in contact lens wearers, and approximately 25,000 contact lens wearers in the United States are infected by P. aeruginosa each year. In addition, according to statistics of the Centers for Disease Control and Prevention (CDC) in the United States, 4 of every 1,000 admitted patients suffer from P. aeruginosa infection. Although P. aeruginosa infection can be treated, the antibiotic resistance of this microorganism increases with the application of antibiotics, the efficacy of treatment is quickly reduced, and it is necessary to change the drug combination and treatment regimen. Data show that P. aeruginosa isolated in an intensive care unit (ICU) of the United States has drug resistance of 51.6% to ciprofloxacin, 31.4% to piperacillin/tazobactam, 38% to imipenem, and 23.6% to ceftazidime. However, P. aeruginosa isolated in an intensive care unit (ICU) of the Europe has drug resistance of 37% to 70% to aminoglycosides, 57% to ceftazidime, 53% to piperacillin/tazobactam, 56% to ciprofloxacin, and 52% to imipenem. Therefore, the treatment and control of serious infection of drug-resistant P. aeruginosa becomes critical.

Typical AMPs reported so far each generally consist of 10 to 100 amino acid residues, and these typical AMPs each include many basic amino acids and are generally amphipathic.

It has been reported that the host defense peptide PGLa extracted from a secretion of a frog has a prominent killing effect on Helicobacter pylori (H. pylori) (gram-negative bacteria). The natural AMP TP4 isolated from the fish body and the natural AMP LL-37 isolated from the human body also have prominent killing effects on H. pylori. These findings show that AMPs have a particularly excellent therapeutic effect for H. pylori, and can also be added as bacteriostatic additives to daily necessities, thereby helping people well stay away from H. pylori. However, the existing AMPs have weak antimicrobial activity, and many of the AMPs do not have broad-spectrum antimicrobial activity.

In addition, AMPs isolated from animals and plants are diverse and widely distributed, which are found in organisms such as insects, fish, mammals, amphibians, and plants. The applicability of AMPs from different sources has not been systematically studied. In particular, plant AMPs are particularly unstable and easily hydrolyzed by proteolytic enzymes, which greatly reduces the antimicrobial activity of the AMPs.

### SUMMARY

An objective of the present application is to provide an AMP or peptide derivative, and a synthesis method, composition, and application thereof. The present application is intended to solve the problem that AMPs in the prior art have weak antimicrobial activity, are unstable, and do not have broad-spectrum antimicrobial activity.

In order to achieve the above objective of the present application, the present application adopts the following technical solutions:

In a first aspect, the present application provides an AMP or peptide derivative, including at least one selected from the group consisting of the following amino acid sequences:

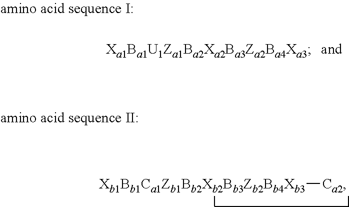

where Xₐ₁, Bₐ₁, U₁, Zₐ₁, Bₐ₂, Xₐ₂, Bₐ₃, Zₐ₂, Bₐ₄, Xₐ₃, X_{b1}, B_{b1}, Cₐ₁, Z_{b1}, B_{b2}, X_{b2}, B_{b3}, Z_{b2}, B_{b4}, X_{b3}, and Cₐ₂ each are independently an amino acid.

The AMP or peptide derivative provided by the present application can specifically combine with a lipid structure of a cell wall/membrane to destroy the microbial wall/membrane, thereby killing microorganisms and cancer cells.

In a second aspect, the present application provides a substitute for an AMP or peptide derivative, where the substitute is obtained by substituting at least one amino acid in the amino acid sequence I and/or the amino acid sequence II in the AMP or peptide derivative of the present application.

The substitute provided in the present application can retain the activity of the AMP or peptide derivative, and is also included in the content of the present application. The AMP or peptide derivative is subjected to amino acid substitution to improve the antimicrobial activity and broad-spectrum antimicrobial activity of the AMP or peptide derivative.

In a third aspect, the present application provides an AMP or peptide derivative composition, at least including the AMP or peptide derivative described above and at least one pharmaceutically acceptable carrier.

The antimicrobial composition provided in the third aspect of the present application is prepared by mixing the AMP or peptide derivative provided in the present application with a solvent. The AMP or peptide derivative can be mixed with most solvents.

In a fourth aspect, the present application provides a preparation method of the antimicrobial composition, including the following step: mixing the AMP or peptide derivative with a solvent.

In the preparation method of the antimicrobial composition of the present application, because the AMP or peptide derivative can exist stably in most solvents, after the AMP or peptide derivative is mixed with the solvent, the antimicrobial activity of the AMP or peptide derivative can be retained, and the stability of the AMP or peptide derivative for peptidase or protein degradation is ensured.

In a fifth aspect, the present application provides an application of the AMP or peptide derivative composition of the present application in packaging, food processing terminal, clothing, medical supplies, medical instruments, personal hygiene products, disinfectants, detergents, anti-infective drugs, anti-inflammatory drugs, and infinite cell proliferation-inhibiting drugs.

The AMP or peptide derivative composition of the present application is widely used because the AMP or peptide derivative has the functions of killing microorganisms and tumor cells, anti-coagulation, and anti-inflammation. The present application mainly has the following four advantages:
1. The AMP or peptide derivative can be combined with or incorporated into packaging, clothing, medical supplies, medical instruments, and personal hygiene materials to make them have an antimicrobial function, or can be used as a preservative for other materials easily degraded by microorganisms.
2. The AMP or peptide derivative can be prepared into a disinfectant, a detergent, an anti-infective drug, or anti-inflammatory drug, thereby playing a sterilization, anti-infection, or anti-inflammation role.
3. The AMP or peptide derivative composition can be prepared into a coating and applied to a food processing terminal, which can reduce the microorganisms on a surface of the food processing terminal to reduce the risk of food poisoning.
4. The AMP or peptide derivative can kill cells and limit cell proliferation, and thus can be used for anti-tumor treatment and local beauty such as wart and spot removal.

### BRIEF DESCRIPTION OF THE DRAWINGS

FIG. 1 is a statistical histogram illustrating the test results of inhibition of an AMP or peptide derivative in an embodiment of the present application on Escherichia coli (E. coli);

FIG. 2 shows the test results of inhibition of an AMP or peptide derivative in an embodiment of the present application on E. coli:

FIG. 3 shows the test results of inhibition of an AMP or peptide derivative in an embodiment of the present application on wild-type (WT) Staphylococcus aureus (S. aureus):

FIG. 4 shows the test minimal inhibitory concentration (MIC) results of inhibition of an AMP or peptide derivative in an embodiment of the present application on P. aeruginosa:

FIG. 5 shows the test results of killing of an AMP or peptide derivative in an embodiment of the present application on RNA viruses:

FIG. 6 shows the test results of killing of an AMP or peptide derivative in an embodiment of the present application on leukemia lymphocytes (Jurkat cell line):

FIG. 7 shows the test results of killing of an AMP or peptide derivative in an embodiment of the present application on leukemia cells (K562 cell line):

FIG. 8 shows the test results of killing of an AMP or peptide derivative in an embodiment of the present application on liver cancer cells; and

FIG. 9 shows the test results of influence of an AMP or peptide derivative in an embodiment of the present application on red blood cells (RBCs).

### DETAILED DESCRIPTION OF THE EMBODIMENTS

To make the to-be-solved technical problems, technical solutions, and beneficial effects of the present application clear, the present application is further described in detail below with reference to embodiments. It should be understood that the specific embodiments described herein are merely intended to explain the present application, rather than to limit the present application.

In the present application, the term “and/or” describes an association relationship between associated objects, and indicates three types of relationships. For example, “A and/or B” may indicate that A exists alone, A and B coexist, or B exists alone. “A” and “B” each may be singular or plural. The character “/” generally indicates that the associated objects are in an “or” relationship.

In the present application, the term “at least one” refers to one or more, and the term “multiple” refers to two or more. The term “at least one of the following items” or similar expression refers to any combination of these items, including any combination of single items or plural items. For example, “at least one of a, b, or c” or “at least one of a, b, and c” can indicate: a, b, c, a-b (namely, a and b), a-c, b-c, or a-b-c, where a, b, and c may each be a single or plural item.

It should be understood that, in various embodiments of the present application, sizes of sequence numbers of the above processes do not imply an order of execution, and some or all steps may be executed in parallel or sequentially. The order of executing the processes should be determined based on their functions and internal logic, and should not constitute any limitation to the implementation processes of the embodiments of the present application.

The terms in the embodiments of the present application are merely used to describe the specific embodiments, and are not intended to limit the present application. Unless otherwise specified in the context, terms in a singular form, such as “a”, “the”, and “this”, in the embodiments and appended claims of the present application include plural forms.

The weight of related components mentioned in the specification of the embodiments of the present application may not only refer to the specific content of each component, but also indicate a proportional relationship between the weights of the components. Therefore, as long as the content of related components is scaled up or reduced according to the specification of the embodiments of the present application, results are within the scope disclosed in the specification of the embodiments of the present application. Specifically, the mass mentioned in the specification of the embodiments of the present application may be in a mass unit known in the chemical industry, such as μg, mg, g, and kg.

The terms “first” and “second” are used only for description to distinguish objects such as substances from each other, and should not be construed as indicating or implying relative importance or implying the number of indicated technical features. For example, without departing from the scope of the embodiments of the present application, a first XX may also be referred to as a second XX, and similarly, the second XX may also be referred to as the first XX. Thus, features defined with “first” and “second” may explicitly or implicitly include one or more of the features.

In a first aspect, an embodiment of the present application provides an AMP or peptide derivative, including at least one selected from the group consisting of the following amino acid sequences:

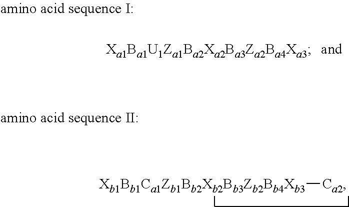

where, Xₐ₁, Bₐ₁, U₁, Zₐ₁, Bₐ₂, Xₐ₂, Bₐ₃, Zₐ₂, Bₐ₄, Xₐ₃, X_{b1}, B_{b1}, Cₐ₁, Z_{b1}, B_{b2}, X_{b2}, B_{b3}, Z_{b2}, B_{b4}, X_{b3}, and Cₐ₂ each are independently an amino acid. In the amino acid sequence II, a cyclic bond is formed between Cₐ₁ and Cₐ₂; and the amino acid includes a natural amino acid (NAA) and/or an unnatural amino acid (UAA). The AMP or peptide derivative provided by the present application can combine with a lipid structure of a cell wall/membrane to destroy the microbial wall/membrane, thereby killing microorganisms and tumor cells.

In an embodiment, on the basis of the above embodiments, in order to enhance the antimicrobial activity of the AMP or peptide derivative, Xₐ₁, Xₐ₂, Xₐ₃, X_{b1}, X_{b2}, and X_{b3} each are independently a positively-charged residue; and/or Bₐ₁, Bₐ₂, Bₐ₃, Bₐ₄, B_{b1}, B_{b2}, B_{b3}, and Bb each are independently a hydrophobic side chain-containing residue; and/or U₁ is one selected from the group consisting of Gly (G-glycine), Pro (P-proline), Cys (C-cysteine), and Cys(R), where R represents a protective group for a Cys disulfide bond; and/or Zₐ₁, Zₐ₂, Z_{b1}, and Z_{b2} each are independently a non-polar residue; and/or, Cₐ₁ and Ca each are independently one selected from the group consisting of Cys (C-cysteine) and Cys (R): in the amino acid sequence I, Xₐ₁ or Xₐ₃ represents an N-terminus, Xₐ₁ Or Xₐ₃ represents a C-terminus; and in the amino acid sequence II, X_{b1} and/or B₂ represent(s) a N-terminus, and Cₐ₂ represents a C-terminus. Since the AMP or peptide derivative of the present application includes a hydrophobic side chain-containing residue, a positively-charged residue, and a non-polar residue, chains of the AMP or peptide derivative provided in this embodiment may form a typical amphoteric helix structure. The chargeability and amphoteric helix structure of the AMP or peptide derivative allow the AMP or peptide derivative to combine with a lipid structure of a cell wall/membrane to destroy the microbial wall/membrane, thereby killing microorganisms and tumor cells.

In an embodiment, the amino acid sequences are as follows:

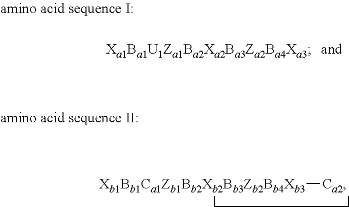

where Bₐ₁, U₁, Zₐ₁, Bₐ₂, Bₐ₃, Zₐ₂, Bₐ₄, B_{b1}, Cₐ₁, Z_{b1}, B_{b2}, B_{b3}, Z_{b2}, B_{b4}, and Cₐ₂ each are independently an amino acid; and Xₐ₁, Xₐ₂, Xₐ₃, X_{b1}, X_{b2}, and X_{b3} each are independently a positively-charged residue. A positively-charged peptide chain is easy to combine with a negatively-charged cell membrane.

In an embodiment, the amino acid sequences are as follows:

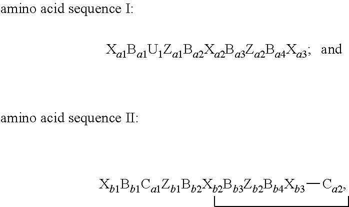

where Bₐ₁, Zₐ₁, Bₐ₂, Bₐ₃, Zₐ₂, Bₐ₄, B_{b1}, Cₐ₁, Z_{b1}, B_{b2}, B_{b3}, Z_{b2}, B_{b4}, and Cₐ₂ each are independently an amino acid: Xₐ₁, Xₐ₂, Xₐ₃, X_{b1}, X_{b2}, and X_{b3} each are independently at least one selected from the group consisting of positively-charged amino acid residues: Bₐ₁, Bₐ₂, Bₐ₃, Bₐ₄, B_{b1}, B_{b2}, B_{b3}, and B_{b3} each are independently at least one selected from the group consisting of hydrophobic side chain-containing amino acid residues; and U₁ is at least one selected from the group consisting of Gly, Pro, Cys, and Cys (R). A positively charged peptide chain is easy to combine with a negatively-charged cell membrane; and the hydrophobic side chain-containing residue can balance the water solubility and chargeability of the AMP or peptide derivative, thereby ensuring the antimicrobial activity of the AMP or peptide derivative.

In an embodiment, the amino acid sequences are as follows:

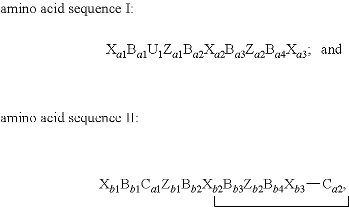

where Cₐ₁ and Cₐ₂ each are independently an amino acid: Xₐ₁, Xₐ₂, Xₐ₃, X_{b1}, X_{b2}, and X_{b3} each are independently a positively-charged residue: Bₐ₁, Bₐ₂, Bₐ₃, Bₐ₄, B_{b1}, B_{b2}, and B_{b3} each are independently at least one selected from the group consisting of hydrophobic side chain-containing amino acid residues: U₁ is one selected from the group consisting of Gly, Pro, Cys, and Cys (R), where R represents a protective group for a Cys disulfide bond; and Zₐ₁, Zₐ₂, Z_{b1}, and Z_{b2} each are independently at least one selected from the group consisting of non-polar amino acid residues. A positively-charged peptide chain is easy to combine with a negatively-charged cell membrane; and the non-polar amino acid residue and the hydrophobic side chain-containing residue can balance the water solubility and chargeability of the AMP or peptide derivative, thereby ensuring the antimicrobial activity of the AMP or peptide derivative.

In an embodiment, the amino acid sequences are as follows:

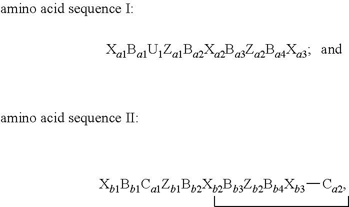

where Xₐ₁, Xₐ₂, Xₐ₃, X_{b1}, X_{b2}, and X_{b3} each are independently a positively-charged residue; Bₐ₁, Bₐ₂, Bₐ₃, Bₐ₄, B_{b1}, B_{b2}, B_{b3}, and B_{b4} each are independently at least one selected from the group consisting of hydrophobic side chain-containing amino acid residues: U₁ is at least one selected from the group consisting of Gly (G-glycine), Pro (P-proline), Cys (C-cysteine), and Cys (R), where R represents a protective group for a Cys disulfide bond: Zₐ₁, Zₐ₂, Z_{b1}, and Z_{b2} each are independently at least one selected from the group consisting of non-polar amino acid residues; Cₐ₁ and Cₐ₂ each are independently at least one selected from the group consisting of Cys (C-cysteine) and Cys (R): in the amino acid sequence I. X₁ and/or B₁ represent(s) an N-terminus, B₁ and/or X₁ represent(s) a C-terminus; and in the amino acid sequence II, X₁ and/or B₂ represent(s) a N-terminus, and Cys represents a C-terminus. Since the AMP or peptide derivative of the present application includes a hydrophobic side chain-containing residue, a positively-charged residue, and a non-polar residue, chains of the AMP or peptide derivative provided in this embodiment may form a typical amphoteric helix structure. The chargeability and amphoteric helix structure of the AMP or peptide derivative allow the AMP or peptide derivative to combine with a lipid structure of a cell wall/membrane to destroy the microbial wall/membrane, thereby killing microorganisms and tumor cells.

In an embodiment, on the basis of the above embodiments, in order to improve the stability of the AMP or peptide derivative, Xₐ₁, X₂₂, Xₐ₃, X_{b1}, X_{b2}, X_{b3}, and B_{b1} each are independently one, two, or three selected from the group consisting of Arg (R-arginine), His (H-histidine), Lys (K-lysine), Orn (ornithine), Har (homoarginine), Dab (2,4-diaminopropionic acid), and unnatural amino acids (UAAs). The Arg (R-arginine), His (H-histidine), Lys (K-lysine), Orn (ornithine), Har (homoarginine), Dab (2,4-diaminopropionic acid), and UAAs are positively-charged residues, and from the overall structure, these residues can combine with other residues to form an amphoteric helix structure, which can increase the antimicrobial activity of the AMP or peptide derivative. If the positively-charged residue in the AMP chain or peptide derivative is too long, the AMP or peptide derivative will undergo charge imbalance and is easy to become unstable. Therefore, in order to ensure the stability of the peptide or peptide derivative for peptidase or protein degradation, there is one, two, or three positively-charged residues, and/or Bₐ₁, Bₐ₂, Bₐ₃, Bₐ₄, B_{b1}, B_{b2}, and B_{b3} each are independently two, three, or four selected from the group consisting of Ala (A-alanine), Val (V-valine), Ile (I-isoleucine), Leu (L-leucine), Met (M-methionine), and UAAs, where Ala (A-alanine), Val (V-valine), Ile (I-isoleucine), Leu (L-leucine), Met (M-methionine), and UAAs are hydrophobic side chains, and the hydrophobic side chains can balance the water solubility and chargeability of the peptide chain. That is, if the hydrophobic side chain residue in the peptide chain or peptide derivative is too long, the peptide chain is easy to become unstable. Therefore, in order to ensure the stability of the peptide chain for peptidase or protein degradation, there are two, three, or four hydrophobic side chain residues, and/or Zₐ₁, Zₐ₂, Z_{b1}, and Z_{b2} each are independently one selected from the group consisting of Asn (N-aspartic acid), Gln (Q-glutamine), Ser (S-serine), Thr (T-threonine), and UAAs, and each are a non-polar residue, which can balance the water solubility and chargeability of the peptide chain. That is, if the non-polar residue in the peptide chain or peptide derivative is too long, the peptide chain is easy to become unstable. Therefore, in order to ensure the stability of the AMP or peptide derivative for peptidase or protein degradation, there are two, three, or four non-polar residues, and/or a sum of numbers of Xₐ₁ and Bal residues, Xₐ₂ and Bₐ₂ residues, Xₐ₃ and Ba residues, X_{b1} and B_{b1} residues, X_{b2} and B_{b2} residues, X_{b3} and B_{b3} residues, or X_{b3} and B_{b1} residues is 3, 4, 5, 6, or 7 and preferably 3 or 4. The longer the peptide chain, the better the activity of the AMP or peptide derivative of the present application. However, considering the cost of synthesis and the stability of the peptide chain, a sum of numbers of Xₐ₁ and Ba residues, Xₐ₂ and Bₐ₂ residues, Xₐ₃ and Bₐ₃ residues, X_{b1} and B_{b1} residues, X_{b2} and B_{b2} residues, X_{b3} and B_{b3} residues, or X_{b4} and B_{b4} residues is preferably 3 and 4. The amino acid sequence and/or a part of the amino acid sequence of the AMP of the present application are/is composed of 8 to 25 amino acids derived from a virus.

In an embodiment, the amino acid sequences are as follows:

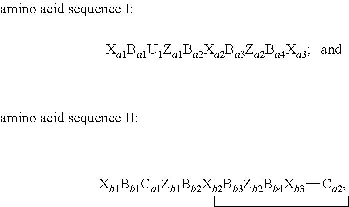

where Zₐ₁, Zₐ₂, Z_{b1}, and Z_{b2} each are independently an amino acid, and Xₐ₁, Xₐ₂, Xₐ₃, X_{b1}, X_{b2}, and X_{b3} each are independently one, two, or three selected from the group consisting of positively-charged Arg. His, Lys, Orn, Har, Dab, and UAAs: Bₐ₁, Bₐ₂, Bₐ₃, Bₐ₄, B_{b1}, B_{b2}, B_{b3}, and Bb each are independently a hydrophobic side chain-containing residue; and/or U₁ is one selected from the group consisting of Gly, Pro, Cys, and Cys (R), where R represents a protective group for a Cys disulfide bond; and Cₐ₁ and Cₐ₂ each are independently one selected from the group consisting of Cys and Cys (R). The positively-charged residue can combine with other residues to form an amphoteric helix structure, which increases the antimicrobial activity of the AMP or peptide derivative. In addition, if the positively-charged residue in the AMP chain or peptide derivative is too long, the AMP or peptide derivative will undergo charge imbalance and is easy to become unstable. Therefore, in order to ensure the stability of the peptide or peptide derivative for peptidase or protein degradation, there is one, two, or three positively-charged residues.

In an embodiment, the amino acid sequences are as follows:

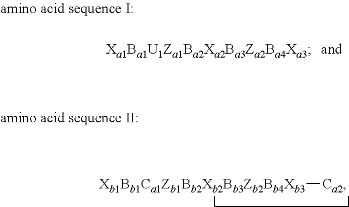

where Xₐ₁, Xₐ₂, Xₐ₃, X_{b1}, X_{b2}, and X_{b3} each are independently one, two, or three selected from the group consisting of positively-charged Arg, His, Lys, Orn, Har, Dab, and UAAs: Bₐ₁, Bₐ₂, Bₐ₃, Bₐ₄, B_{b1}, B_{b2}, B_{b3}, and Bb each are independently two, three, or four selected from the group consisting of hydrophobic side chain-containing Ala, Val, Ile, Leu, Met, and UAAs; and/or U₁ is one selected from the group consisting of Gly, Pro, Cys, and Cys (R), where R represents a protective group for a Cys disulfide bond: Zₐ₁, Zₐ₂, Z_{b1}, and Z_{b2} each are independently at least one selected from the group consisting of non-polar residues; and/or Cₐ₁ and Cₐ₂ each are independently one selected from the group consisting of Cys and Cys (R). The positively-charged residue can combine with other residues to form an amphoteric helix structure, which increases the antimicrobial activity of the AMP or peptide derivative. In addition, if the positively-charged residue in the AMP chain or peptide derivative is too long, the AMP or peptide derivative will undergo charge imbalance and is easy to become unstable. Therefore, in order to ensure the stability of the peptide or peptide derivative for peptidase or protein degradation, there is one, two, or three positively-charged residues. The hydrophobic side chain can balance the water solubility and chargeability of the peptide chain, that is, if the hydrophobic side chain residue in the peptide chain or peptide derivative is too long, the peptide chain is easy to become unstable. Therefore, in order to ensure the stability of the peptide chain for peptidase or protein degradation, there are two, three, or four hydrophobic side chain residues.

In an embodiment, the amino acid sequences are as follows:

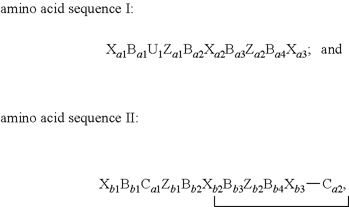

where Xₐ₁, Xₐ₂, Xₐ₃, X_{b1}, X_{b2}, and X_{b3} each are independently one, two, or three selected from the group consisting of positively-charged Arg, His, Lys, Orn, Har, Dab, and UAAs: Bₐ₁, Bₐ₂, Bₐ₃, Bₐ₄, B_{b1}, B_{b2}, B_{b3}, and B_{b4} each are independently two, three, or four selected from the group consisting of hydrophobic side chain-containing Ala, Val, Ile, Leu, Met, and UAAs: Zₐ₁, Zₐ₂, Z_{b1}, and Z_{b2} each are independently one selected from the group consisting of non-polar Asn, Gln, Ser, Thr, and UAAs: U₁ is one selected from the group consisting of Gly, Pro, Cys, and Cys (R), where R represents a protective group for a Cys disulfide bond; and Cₐ₁ and Cₐ₂ each are independently one selected from the group consisting of Cys and Cys (R). The positively-charged residue can combine with other residues to form an amphoteric helix structure, which increases the antimicrobial activity of the AMP or peptide derivative. In addition, if the positively-charged residue in the AMP chain or peptide derivative is too long, the AMP or peptide derivative will undergo charge imbalance and is easy to become unstable. Therefore, in order to ensure the stability of the peptide or peptide derivative for peptidase or protein degradation, there is one, two, or three positively-charged residues. The hydrophobic side chain can balance the water solubility and chargeability of the peptide chain, that is, if the hydrophobic side chain residue in the peptide chain or peptide derivative is too long, the peptide chain is easy to become unstable. Therefore, in order to ensure the stability of the peptide chain for peptidase or protein degradation, there are two, three, or four hydrophobic side chain residues, which can balance the water solubility and chargeability of the peptide chain. That is, if the non-polar residue in the peptide chain or peptide derivative is too long, the peptide chain is easy to become unstable. Therefore, in order to ensure the stability of the AMP or peptide derivative for peptidase or protein degradation, there are two, three, or four non-polar residues.

In some embodiments, the Cys disulfide bond includes acetamidomethyl (Acm), methyl methanethiosulfonate, or other Cys protective groups, and Cys (R) indicates a protective group with a Cys disulfide bond, where Cₐ₂ is a terminal group of the AMP or peptide derivative and Cal is located in the middle of the AMP or peptide derivative. Therefore, a cyclic chain structure can be formed inside a molecule of the AMP or peptide derivative, which can enhance the stability of the AMP, extend the sterilization time of the AMP, and make the AMP or peptide derivative have strong antimicrobial activity.

In some embodiments, the amino acid sequence(s) I and/or II have/has at least one amino acid modified. In practical applications, the stability and antimicrobial activity of the AMP or peptide derivative are very important. The amino acid in the AMP or peptide derivative of the present application can be modified to change the performance of a peptide chain, improve the spectroscopic performance of the AMP or peptide derivative, improve the stability of the AMP or peptide derivative for peptidase or protein degradation, and increase the broad-spectrum antimicrobial activity of the AMP or peptide derivative.

In an embodiment, the modification includes phosphorylation, halogenation, acetylation, cyclization, and terminal capping, where the cyclization includes at least one selected from the group consisting of cyclization caused by a disulfide bond, head-to-tail cyclization, and cyclization of side groups in an internal structure; and the terminal capping includes at least one selected from the group consisting of C-terminal amidation, N-terminal acetylation, and N-terminal phospholipidation. The modification such as cyclization of the amino acid can increase the stability of the peptide chain, and the phosphorylation of the amino acid can increase the antimicrobial activity of the AMP or peptide derivative.

In some embodiments, the amino acid sequence(s) I and/or II include(s) at least one motif. The motif is often linked to a given terminal amino acid sequence (N-terminus and/or C-terminus) and can also be directly inserted into a given amino acid sequence, and the motif generally does not independently serve as an amino acid sequence terminus. The linking or insertion of the motif to the AMP or peptide derivative can change the performance of the peptide chain, increase the antimicrobial activity of the peptide chain, and enhance the stability of the AMP or peptide derivative for peptidase or protein degradation.

In some embodiments, the AMP or peptide derivative is a multimer formed by the amino acid sequence I and/or the amino acid sequence II, including at least one selected from the group consisting of a dimer and a tetramer, which are common multimers. The AMP monomer and/or multimer can be conjugated with at least one drug or antibody to produce an antimicrobial drug, and the at least one drug or antibody mainly includes a small-molecule chemical synthetic drug, a large-molecule antibody, and a large-molecule drug synthesized by a biological combination technology. The conjugation of the multimer formed by the AMP or peptide derivative of the present application with a drug or antibody molecule to produce an antimicrobial drug can be used for the modification of the drug or antibody.

In some embodiments, the AMP or peptide derivative includes amino acid sequences shown in SEQ ID NO: 1 to SEQ ID NO: 39.

In some embodiments, the AMP or peptide derivative has a nanostructure, where a peptide chain and a peptide derivative thereof, or multiple peptide chains or a polypeptide chain and peptide derivatives thereof may form a nanostructure; and a common nanostructure is at least one selected from the group consisting of a micelle, a vesicle, a nanotube, and a nanoribbon. The formation of a nanostructure by the peptide chain and a peptide derivative thereof facilitates the stable presence of the peptide chain and a peptide derivative thereof in a solution and the transfer of the peptide chain and a peptide derivative thereof in the solution.

In a second aspect, the present application provides a substitute for the AMP or peptide derivative, which is obtained by substituting an amino acid in the amino acid sequence I or II described above and is also included in the content of the present application, such as substituting an L-amino acid with a corresponding D-amino acid or a UAA. The AMP or peptide derivative is subjected to amino acid substitution to improve the antimicrobial activity and broad-spectrum antimicrobial activity of the AMP or peptide derivative.

In a third aspect, the present application provides an AMP or peptide derivative composition, including the AMP or peptide derivative and/or the substitute, and at least one pharmaceutically acceptable carrier and/or solvent. The AMP or peptide derivative provided in the present application may be combined with each of most pharmaceutically acceptable carriers to prepare a pharmaceutical composition, and may also be mixed with each of most solvents to prepare a composition.

In some embodiments, the composition further includes at least one selected from the group consisting of an excipient, an isotonic agent, an absorption retarder, and a basal polymer; and the AMP or peptide derivative can be mixed with these substances to prepare various antimicrobial drugs or solvents.

In some embodiments, the excipient is at least one selected from the group consisting of water, a salt, a phosphate, glucose, glycerol, and ethanol, which are common medical reagents. The mixing of the AMP or peptide derivative with these reagents is conducive to the promotion and application of the AMP or peptide derivative in the present application.

In some embodiments, the isotonic agent is selected from the group consisting of a sugar, a polyol, and sodium chloride, and the isotonic agent can maintain the peptide activity. After being mixed with the isotonic agent, the AMP or peptide derivative can easily combine with an organism.

In some embodiments, the polyol is selected from the group consisting of mannitol and sorbitol; and after the polyol is mixed with the AMP or peptide derivative, the polyol can maintain the activity of the AMP or peptide derivative and make the AMP or peptide derivative easily combine with an organism.

The pharmaceutically acceptable carrier is an auxiliary substance with the minimum amount, and the pharmaceutically acceptable carrier is selected from the group consisting of a wetting agent, an emulsifying agent, a preservative, and a buffer. After the pharmaceutically acceptable carrier is mixed with the AMP or peptide derivative to produce a composition, the activity of the AMP or peptide derivative can be retained, and the AMP or peptide derivative can easily combine with an organism.

In an embodiment, the AMP or peptide derivative is combined with a basal polymer to prepare an aqueous or non-aqueous solution, and the aqueous or non-aqueous solution may be applied to a surface of an object to form an antimicrobial protective layer.

In addition, the above basal polymer may be in various forms or may be composed of various components. The basal polymer includes, but is not limited to, an extract, a film, a membrane, a laminate, a knitted fabric, a woven fabric, a non-woven fabric, a fiber, a filament, a yarn, a granule, a coating, and/or a foam. The present application also includes various products prepared by the above substances and the AMP, including, but not limited to, an injection-molded product, an extrusion-molded product, a blow-molded product, a thermoformed product, a solution coating, a blown film, a knitted product, a woven product, and a textile product.

In a fourth aspect, the present application provides a preparation method of the AMP or peptide derivative composition, including the following step: mixing the AMP or peptide derivative with the solvent.

In an embodiment, when the AMP or peptide derivative is mixed with the basal polymer to obtain a mixture, in order to ensure thorough mixing, the mixing is conducted at −10° C. to 150° C. for 0.1 min and 5,760 min. When the mixing is conducted at 25° C. to 80ºC for 1 min to 1,440 min, a prominent mixing effect can be achieved.

In some embodiments, the pharmaceutically acceptable carrier, the excipient, the solvent, the dispersion medium, the isotonic agent, the absorption retarder, or the basal polymer can be pretreated, and the pretreatment includes, but is not limited to, at least one selected from the group consisting of oxidation, reduction, hydrolysis, plasma, and radiation. The pretreatment may improve the environment of the AMP or peptide derivative, thereby improving the stability of the AMP or peptide derivative for peptidase or protein degradation.

In some embodiments, an effective concentration range of the AMP or peptide derivative when mixed with the above substances is in a range of 1 μg to 20 g wt % or in a range of 5 pmol to 2 mol. The AMP or peptide derivative composition needs to be preserved at a temperature controlled in a range of −20° ° C. to 25° C. to ensure the antimicrobial activity of the AMP or peptide derivative. In addition, the temperature needs to be controlled in a range of −4° C. to 4° C. to ensure the biological activity of the AMP.

In a fifth aspect, the present application provides an application of the AMP or peptide derivative composition in any one selected from the group consisting of the following:
packaging, food processing terminal, clothing, medical supplies, medical instruments, personal hygiene products, disinfectants, detergents, anti-infective drugs, anti-inflammatory drugs, and infinite cell proliferation-inhibiting drugs. The AMP or peptide derivative composition can be widely used as a drug or non-pharmaceutical product in human beings, animals (including wild animals, livestock, and companion animals), plants (including crops), aquatic and aquaculture animals, farmed poultry, or the like. The concept definitions hereinafter for human beings, animals, and plants are the same as the concept definitions for human beings, animals (including wild animals, livestock, and companion animals), plants (including crops), aquatic and aquaculture animals, and farmed poultry mentioned above, which are the same below.

In some embodiments, in the application of the composition in packaging, the AMP or peptide derivative may be combined with or incorporated into a packaging material, or may be used as a preservative for another material easily degraded by microorganisms, where the packaging material includes, but is not limited to, a packaging film for a meat product, a liner, an absorbent pad, a tray, a container component, a cap, a lid, an adhesive, and an applicator. The packaging can be in any form suitable for special applications, such as aluminum cans, boxes, bottles, glass jars, bags, cosmetic packaging, and closed tubes. The packaging can be fabricated by a process, including, but not limited to, injection-molding, extrusion-molding, blow-molding, thermoforming, solution coating, and film blowing.

The packaging is also suitable for the packaging of prescription/over-the-counter (OTC) drugs and health care/hygiene products, such as bottles, tips, applicators, and lids for capsules, tablets, solutions, emulsifying agents, detergents, powders, shampoos, conditioners, deodorants, and antiperspirants. The packaging is also suitable for the packaging of applicators, such as lipstick, lip balm, and overprint varnish, and for the packaging of eye cosmetics, such as mascara, eyeliner, eye shadow, spray powder, bath powder, blush, foundation, and lotion. The applicator can be used at different parts and surfaces of an organism to greatly reduce or eliminate the growth of bacteria at the parts and surfaces of the organism. In addition, other forms of packaging composition can be adopted, such as drinking water bottlenecks for carrying liquids, solutions, or suspensions, replaceable lids, non-replaceable lids, and food or drug-dispensing systems: food and beverage delivery systems; and baby bottles, lids, and pacifiers. The packaging also includes a glue-drop chaotropic agent or a liquid-drop ejector.

In some embodiments, the composition described above can be used in food processing terminal, or can be used to prepare a temporary or permanent coating for a food surface, such as food additives to replace antibiotics: food processing instruments, food conveyor belt assembly and components, and an instrument assembly and components for mixing, grinding, crushing, rolling, granulating, and squeezing food; and an instrument assembly and components for cutting and slicing food. If the above instrument has a metal surface, the metal surface must be coated with a layer of a functional polymer, and the polymer is the polymer with the AMP or peptide derivative in the present application. The AMP or peptide derivative composition can be prepared into a coating and applied to a food processing terminal, which can reduce the microorganisms on a surface of the food processing terminal to reduce the risk of food poisoning.

In some embodiments, when the composition described above is used in clothing, such clothing has bactericidal and bacteriostatic functions, such as swimming suits, underclothes, shoe components (such as woven or non-woven liners or insoles), sports protective pads, and children's clothing. The present application also includes protective medical clothing or isolation supplies such as protective clothing, masks, gloves, slippers, boots, head covers, or draping curtains.

In some embodiments, for the application of the composition described above in medical supplies and instruments, the AMP or peptide derivative may be combined with or incorporated into medical supplies or medical materials, may be coated on a surface of a medical instrument to form an AMP or peptide derivative layer, or may be prepared into a spray for disinfecting medical supplies and instruments. The medical supplies and instruments include human and animal implants, such as bandages, adhesives, gauze strips, gauze pads, syringe holders, peripheral venous or central venous cannulae (the cannulae can be made of polyurethane (PU) or silica gel), urinary catheterostomy ports, orthopedic orthoses, orthopedic needles, pacemaker leads, defibrillator leads, ear canal shunts, vascular stents, orthopedic implants, ear, nose, and throat (ENT) implants, implantable pumps, hernia patches, and related plates, screws, blood bags, topical blood pumps, infusion systems, cardiopulmonary instruments, dialysis instruments, artificial skin, artificial hearts, ventricular assist devices (VADs), hearing aids, vascular grafts, pacemaker components, hip implants, knee implants, and dental implants.

In some embodiments, for the application of the composition described above in personal hygiene products, the AMP or peptide derivative may be combined with or incorporated into personal hygiene products, such that the personal hygiene products have bactericidal and bacteriostatic functions, such as diapers, toilet pads, sanitary napkins, sports pads, tampons, and accessory instruments using the same; and hygiene and health care products such as antimicrobial wet wipes, baby wet wipes, personal wet wipes, cosmetic wet wipes, diapers, and medical wet wipes (such as antibiotic-containing wet wipes or pads, acne-treating drugs, hemorrhoid-treating drugs, antipruritics, anti-inflammatory drugs, and preservatives).

In some embodiments, when the composition described above is used in an article in direct contact with the oral cavity, the AMP or peptide derivative is non-toxic to the human body and thus can be in direct contact with the oral cavity to achieve the purpose of oral sterilization and bacteriosis, where the article in direct contact with the oral cavity can prevent infant skin from infection, and is a baby bottle, a pacifier, a dental instrument, an elastic band, a denture, a cup, a water glass, a toothpaste, and a teething toys, for example. Pediatric articles mainly intended to prevent microbial infection are also in the protection scope of the present application, such as baby bottles, children's books, plastic scissors, toys, diaper pails, and containers for holding clean wet wipes.

In some embodiments, when the composition described above is used in household products, in order to prevent bacteria from entering through the mouth, the AMP or peptide derivative may be combined with or incorporated into a household material, where the household products include telephones, mobile phones, fiber fillings, bedding, window treatments, carpet-cleaning treatments, foam cushions on the back of floor mats or carpet mats, upholstery articles (such as foam cushions), non-woven dry paper, softening agent-containing paper, car wet wipes, household cleaning wet wipes, countertop wet wipes, shower curtains, shower curtain fabrics, towels, face towels, rags, mops, tablecloths, wallpaper, and countertops.

In some embodiments, for the application of the composition described above in a disinfectant, in order to avoid cross-infection, the AMP or peptide derivative of the present application may be prepared into a disinfectant, and the disinfectant can be used for air sterilization in an environment of an enclosed space, a plane, a train, a theater, a theater, or the like. For example, the disinfectant can be used for the disinfection of a medical endoscope. The AMP or peptide derivative can reduce or prevent the formation of a biofilm on a surface of an independent membrane, and the independent membrane includes a pervaporation membrane, a dialysis membrane, a reverse osmosis membrane, an ultrafiltration membrane, a microfiltration membrane, or the like.

In some embodiments, for the application of the composition described above in a detergent, the detergent has a bactericidal effect, and after an article is cleaned with the detergent, the article needs to be cleaned with water and is optimally cleaned with deionized water. The cleaned article can be selectively dried, where the drying can be drying with ambient air, drying in an oven, and forced drying with air; and the drying is conducted at 50° C. to 120° C. and preferably 50° ° C. to 100° C., and the drying is conducted for about 15 min to 24 h.

In addition, the treatment of the articles with the AMP or AMP composition is conducted in a process before industrial production, after industrial production, or in industrial production. For example, when the AMP is used to prepare an antimicrobial shower curtain, the AMP is first mixed with a basal polymer, and then a resulting mixture is used to treat a shower curtain. A production process includes, but is not limited to, at least one selected from the group consisting of injection-molding, extrusion-molding, blow-molding, thermoforming, solution coating, and film blowing.

In some embodiments, the composition described above can be used in anti-infective drugs and anti-inflammatory drugs for sterilization to prevent infection, and the infection may be caused by bacteria, viruses, or fungi, including yeast, pathogens, gram-negative and/or gram-positive bacteria, and unicellular or multicellular organisms, and may also be caused by symbiotic or non-symbiotic organisms, pathogenic organisms, and colonic or non-colonic bacteria. Specifically, the AMP or peptide derivative can be used to treat or reduce bacterial, viral, and fungal infections on the skin, and the infections can occur at any part related to the human or animal epithelial system, such as epidermis, dermis, subcutaneous tissue, one or more hair follicles, one or more sebaceous glands, or other parts associated with the skin. In other words, the infections are associated with the skin. Further, the infections can also be associated with a part of vision or a part of hearing, such as a part of the ear, such as tympanic cavity or a part of a hearing system including the tympanic cavity.

In an embodiment, the anti-infective drug may be used to treat infections in humans or animals caused by any one or more bacteria; and the AMP may treat the excessive growth of one or more symbiotic organisms, even if such symbiotic organisms are non-pathogenic or symbiotic organisms beneficial to humans or animals. Some symbiotic organisms may come from the following species: Staphylococcus, Mycobacteria, and Propionibacterium. In addition, other bacterial infections may come from known pathogens, such as Pseudomonas.

In an embodiment, the anti-infective drug may be used to treat infections caused by non-bacterial microorganisms, such as infections caused by one or more fungi (such as yeast). For example, the AMP can be used to treat infections caused by Malassezia furfur (M. furfur). M. furfur is a symbiotic organism, and the excessive growth of M. furfur will cause dandruff, seborrheic dermatitis, Pityriasis versicolor, folliculitis, or the like, which are often treated by controlling the growth of M. furfur, eliminating related inflammation, and controlling secondary infection. M. furfur grows stubbornly, is not easy to treat, and needs to be treated through the combined action of many drugs with a long treatment cycle, and corresponding drugs currently used on the market have toxic and side effects and are extremely expensive. The AMP of the present application may treat infections caused by fungi such as M. furfur in an effective therapeutic concentration range.

In an embodiment, the anti-inflammatory drug can be used to treat inflammation caused by infection, non-infection, or even physical trauma, and the AMP or peptide derivative exhibits the effect of reducing, alleviating, and treating inflammation in experiments. For example, in cells, bacteria, and animals, the AMP can reduce the release of inflammatory factors such as cytokinesis, chemokines, and analogues thereof.

In addition, the inflammation is a physiologically-defined inflammatory response, including redness, swelling, and induction of one or more pro-inflammatory cytokines at a cellular molecular transcription and translation level: induction of one or more cellular signaling pathways associated with inflammation: induction of receptors in cell membranes: cellular infiltration within vascularized tissues; and other manifestations associated with inflammation within the technical scope of the prior art.

In some embodiments, the composition described above can be used in infinite cell proliferation-inhibiting drugs for anti-tumor, where infinitely-proliferating cells (cell lines in vitro and in vivo) include human-constructed cells and cells in disease states such as cancer cells; and the infinitely-proliferating cells also include primary cells such as keratinocytes, microvascular endothelial cells (MVECs), corneal epithelial cells, and dermal fibroblasts. The AMP composition can cause a decrease in pattern-recognition receptors (PRRs), which are cell membrane receptors capable of specifically binding to bacteria. As a result of the above reaction, significant inflammation-relieving effects, such as reduced or disappeared tissue redness and swelling and reduced or disappeared cell penetration, are observed in animals and animal parts.

In some embodiments, the pro-inflammatory cytokines include cytokines associated with inflammation, including, but not limited to, tumor necrosis factor (TNF), interleukin 8, interleukin 1, and interleukin 6. In addition, intracellular signaling pathways include signaling pathways associated with inflammation, including, but not limited to, NFkB and AP-1. The cell membrane-associated receptors include, but are not limited to, PRRs of the TLR receptor family (including TLR-2 and TLR4).

In an embodiment, the therapeutic concentration and time of the AMP depend on many factors, such as disease status, age, gender, body weight, and personal physical conditions; and when the AMP is used in cellular therapy, an effective concentration of the AMP is greater than 0.01 μg/mL.

In an embodiment, the effective concentration is greater than 0.2 μg/mL.

In an embodiment, dosage forms of the anti-infective drug, the anti-inflammatory drug, and the infinite cell proliferation-inhibiting drug each are at least one selected from the group consisting of a liquid, a semi-solid, a creamy solid, an ointment, and a gel. For example, a dosage form for external use may be applied to the skin, hair, and other external parts, and further, under specified conditions, the dosage form for external use can be applied to one or both eyes and can also be prepared into eye drops.

In an embodiment, the AMP or peptide derivative may be prepared into an intravenous injection dosage form or an intratympanic/transtympanic dosage form, and a solvent may be a sterilized isotonic aqueous solution.

In addition, the dosage form of the AMP used medically should be consistent with an administration route, and the administration route includes gastrointestinal administration, intravenous injection, subcutaneous administration, intradermal administration, oral administration, intranasal administration (inhalation), vaginal administration, anal administration, epidermal administration, transmucosal administration, transtympanic administration, intratympanic administration, rectal administration, and other acceptable administration routes. The dosage form of the AMP should match an administration route consistent with pharmaceutical components. For example, the AMP can be administered through intravenous injection, subcutaneous administration, intramuscular administration, transganglion administration, oral administration, intranasal administration, intraaural administration, and subepidermal administration to humans and/or animals, such as livestock, companion animals, aquatic species, and poultry.

In addition, the definition of an amino acid represented by one letter in the present application is consistent with the international standards for amino acid codes and representative sequences (IUPAC-IYUB), as shown in Table 1.

TABLE 1

| | Three-letter | One-letter |
|---|---|---|
| Amino acid | abbreviation | abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Ornithine | Orn | |
| Homoarginine | Har | |
| 2,4-diaminobutyric acid | Dab | |

The present application will be described below with reference to specific examples.

### Examples 1 to 39

The amino acid sequences in Examples 1 to 39 were synthesized by the standard internationally-known solid-phase synthesis, solution-phase synthesis, or recombinant biosynthesis, and synthesized amino acid sequences were shown in Table 2.

TABLE 2

| Example | Amino acid sequence | Coding |
|---|---|---|
| 1 | HAVGNIMHIASAVLVRH | SEQ ID NO: 1 |
| 2 | HKAVGQIMHIASAVLVRH | SEQ ID NO: 2 |
| 3 | HRAMPNMLKIMASLVLAKR | SEQ ID NO: 3 |
| 4 | HAMPNMLRIMASLVLARHK | SEQ ID NO: 4 |
| 5 | HKAVPNMLHIASAVLVRH | SEQ ID NO: 5 |
| 6 | HKAVGNMLKIASAVLVHK | SEQ ID NO: 6 |
| 7 | HAMPNMLRIMASAVLVKR | SEQ ID NO: 7 |
| 8 | HAMGNMLKIMASAVLVRH | SEQ ID NO: 8 |
| 9 | HRAMGQIVKMIATLVLARKH | SEQ ID NO: 9 |
| 10 | HKRAMPSIVKMIATLVLARKH | SEQ ID NO: 10 |
| 11 | RKAMPNMLRIMASAVLVHK | SEQ ID NO: 11 |
| 12 | RHAMGNMLHIMASLVLARKH | SEQ ID NO: 12 |
| 13 | RHAVPNMLRIMASLVLAHKR | SEQ ID NO: 13 |
| 14 | RAMPNMLKIMASAVLVHK | SEQ ID NO: 14 |
| 15 | RAMPNMLRIMASLVLARKH | SEQ ID NO: 15 |
| 16 | RKAVPNMLRIMASAVLVHKR | SEQ ID NO: 16 |
| 17 | RHAMGQMLRIMASLVLARK | SEQ ID NO: 17 |
| 18 | RKHAMPSIVKMIATLVLARKH | SEQ ID NO: 18 |
| 19 | RKHAMGQIVKMIATLVLAHR | SEQ ID NO: 19 |
| 20 | RHAVPNMLRIMASAVLVKHR | SEQ ID NO: 20 |
| 21 | KAVPNIMHIMASAVLVRH | SEQ ID NO: 21 |
| 22 | KRAMPNIVRIMASAVLVRH | SEQ ID NO: 22 |
| 23 | KHAMPNMLRIMASLVLARH | SEQ ID NO: 23 |
| 24 | KAVPNMLRIMASLVLARKH | SEQ ID NO: 24 |
| 25 | KHAVGQIVKMIATLVLAKHR | SEQ ID NO: 25 |
| 26 | KRAMPSIVKMIATAVLVRKH | SEQ ID NO: 26 |
| 27 | KAVPNMLKIMASLVLARH | SEQ ID NO: 27 |
| 28 | KHAVPNMLRIMASLVLAHKR | SEQ ID NO: 28 |
| 29 | KHRAVGNMLKIASLVLARHK | SEQ ID NO: 29 |
| 30 | KAMPNIVRIMASLVLARHK | SEQ ID NO: 30 |
| 31 | 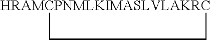 | SEQ ID NO: 31 |
| 32 | 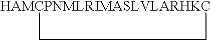 | SEQ ID NO: 32 |
| 33 | 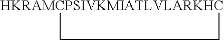 | SEQ ID NO: 33 |
| 34 | 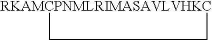 | SEQ ID NO: 34 |
| 35 | 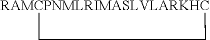 | SEQ ID NO: 35 |
| 36 | 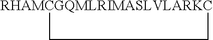 | SEQ ID NO: 36 |
| 37 | 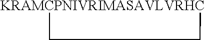 | SEQ ID NO: 37 |
| 38 | 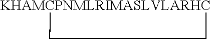 | SEQ ID NO: 38 |
| 39 | 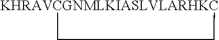 | SEQ ID NO: 39 |

### Performance Test and Result Analysis

(1) E. coli Inhibition Test

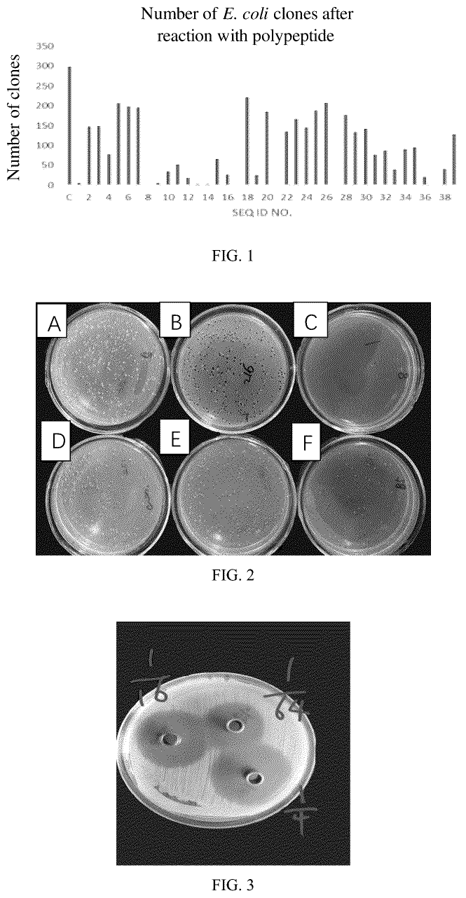

The 39 AMPs synthesized in Examples 1 to 39 each were subjected to an E. coli inhibition test. 30 μL of an E. coli bacterial solution was taken and mixed with 30 μL of each of the AMPs (concentration: 200 μg/mL), and 30 μL of a PBS isotonic solution was adopted to prepare a control; a reaction was allowed at room temperature for 15 min, and a resulting bacterial solution was evenly coated on an agarose plate and cultivated at 37ºC for 12 h; and the number of E. coli bacteria on each plate was counted and recorded. Experimental results were shown in FIG. 1. The experimental results show that the AMPs of SEQ ID NO: 1 to SEQ ID NO: 39 have inhibitory effects on E. coli, and the AMP of SEQ ID NO: 8 exhibits the most significant inhibitory effect.

In order to make the experimental results clearer, 6 experimental samples were taken for control analysis. As shown in FIG. 2, A: control, 251 E. coli clones: B: SEQ ID NO: 5, 236 E. coli clones: D: SEQ ID NO: 6, 226 E. coli clones: F: SEQ ID NO: 7, 216 E. coli clones: C: SEQ ID NO: 8, 1 E. coli clone; and E: SEQ ID NO: 35, 95 E. coli clones, indicating that the AMPs have the effect of killing gram-negative bacteria. In addition, the 1 E. coli clone corresponding to the AMP of SEQ ID NO: 8 indicates that the optimal AMP has an amino acid sequence shown in SEQ ID NO: 8 and has an MIC of 32 μg/mL for E. coli.

(2) Inhibition Zone Test on WT S. aureus

A bacterial solution of clinically isolated WT S. aureus (gram-positive bacteria) was taken and coated on an agarose plate, an Oxford cup for inoculation was placed, and 200 μL of the AMP of SEQ ID NO: 8 was added (concentrations: ¼: 375 μg/mL: 1/16: 93.7 μg/mL; and 1/64: 23.34 μg/mL); and the bacteria were cultivated at 37ºC for 18 h, and then a size of an inhibition zone was measured. The experimental results were as follows: ¼: inhibition zone: 3.95 cm: 1/16: inhibition zone: 3.5 cm; and 1/64: inhibition zone: 3.1 cm, as shown in FIG. 3. The experimental results show that, with the decrease of the concentration of the AMP, the inhibition zone decreases. In addition, the AMP is more sensitive to S. aureus than E. coli, and the AMP of the present application has prominent resistance to gram-positive bacteria.

(3) Determination of MIC for P. aeruginosa (Pseudomonas ATCC27853)

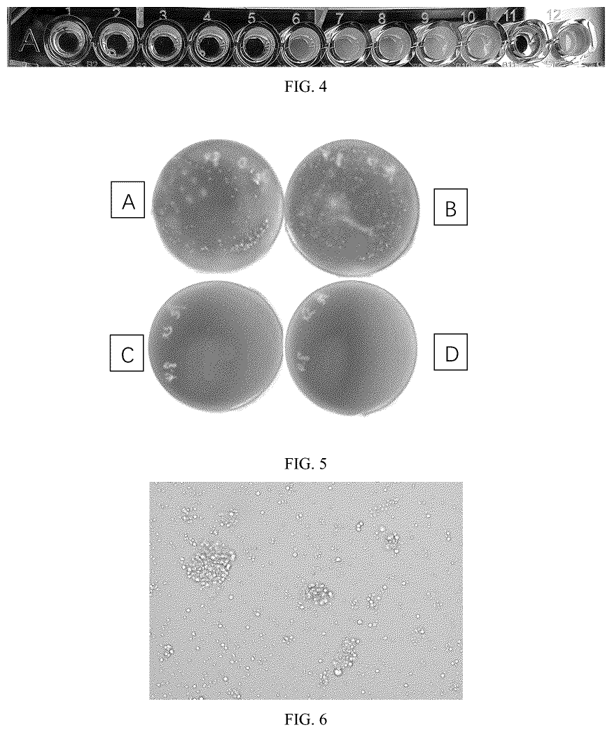

The AMP of SEQ ID NO: 8 was taken at concentrations of 1,024 μg/mL, 512 μg/mL, 256 μg/mL, 128 μg/mL, 64 μg/mL, 32 μg/mL, 16 μg/mL, 8 μg/mL, 4 μg/mL, and 2 μg/mL, and positive and negative controls were set: a sample was mixed with standard P. aeruginosa and then added to 12 wells of a culture plate, and the 12 wells of the culture plate were numbered; and P. aeruginosa was cultivated at 37ºC for 2 d, and then a turbidity degree of the culture plate was observed. As shown in FIG. 4, 1 corresponds to 1,024 μg/mL, 2 corresponds to 512 μg/mL, 3 corresponds to 256 μg/mL, 4 corresponds to 128 μg/mL, 5 corresponds to 64 μg/mL, 6 corresponds to 32 μg/mL, 7 corresponds to 16 μg/mL, 8 corresponds to 8 μg/mL, 9 corresponds to 4 μg/mL, 10 corresponds to 2 μg/mL, 11 corresponds to the positive control, and 12 corresponds to the negative control. Experimental results were shown in FIG. 4. The experimental results show that the AMP has a prominent inhibitory effect on P. aeruginosa, with MIC of 64 μg/mL for P. aeruginosa.

### (4) Inhibition on Oral Bacteria

Mouthwash was taken from the elderly and divided into 4 parts of equal amounts, and the 4 parts each were mixed with a solution of 2 mg/mL SEQ ID NO: 8 at different amounts for 30 min and then evenly coated on plates with sheep blood agar, where A: PBS (control), B: 2 μL, C: 10 μL, and D: 20 μL; and resulting mixtures each were anaerobically cultivated at 37ºC for 4 d, and the number of colonies on each plate was observed. Experimental results were shown in FIG. 5.

Specifically, it was found that Candida albicans (C. albicans) and anaerobic bacteria grew on the control plate, a small number of bacteria grew on the B: 2 μL AMP plate, no oral bacteria grew on the C: 10 μL AMP plate, and no oral bacteria grew on the D: 20 μL AMP plate.

### (5) Killing of the AMP for RNA Viruses

The plasmids pNL4.3Δ, pVSV-G, p-enhancer, and lipo2000 each were used to transfect 293T, then the cells were cultivated at 37° ° C. for two days, a culture supernatant was taken and centrifuged to remove organelle impurities, and 500 μL of a cell supernatant was mixed with an equal amount of 200 μg/mL SEQ ID NO: 8 to allow a reaction for 15 min. In a control group, PBS was used instead of SEQ ID NO: 8. The filter centrifugation (Millipore Amicon Ultra-15) was used to remove the AMP, thereby eliminating a killing effect of the AMP itself on cells. A sample to be tested was used to infect the 293Tcell line, and then the cell line was cultivated at 37° C. for 12 h. The 293T cell line was recovered, and the RT-PCR method was used to determine the infection difference between live viruses for the cells. The control group and the experimental group had the same experimental environment, but the amino acid sequence of SEQ ID NO: 8 of the present application was added in the experimental group.

The results show that the active viral gene copy of the control group is 1.7×10⁷ and the active viral copy of the AMP group is 5×10⁵, indicating that the AMP of the present application has a significant virus-killing effect.

### (6) Killing of the AMP for Tumor Cells

K562 cells were cultivated, a same amount of K562 cells was taken and mixed with 10 ng, 20 ng, and 40 ng of the AMP of SEQ ID NO: 8 to allow a reaction, and apoptotic cells were determined by a flow cytometry (FCM) PI kit. It was found that the cell death was proportional to the content of the AMP, indicating that the AMP could effectively kill K562 cells.

### (7) Killing of the AMP for Leukemia Lymphocytes

Jurkat cell line (leukemia lymphocytes) was taken. 20 μL of a solution of 1 μg/mL AMP of SEQ ID NO: 10 was mixed with 10⁵ cells to allow a reaction for 10 min, and a resulting mixture was centrifuged at 1,000 rpm for 3 min. An experimental image was shown in FIG. 6. Experimental results show that the leukemia lymphocytes clump, and some are broken.

### (8) Killing of the AMP for Leukemia Cells

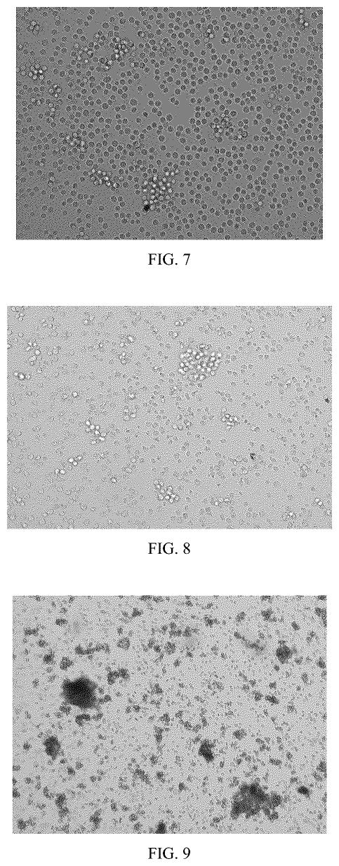

K562 cell line (leukemia cells) was taken. 20 μL of a solution of 1 μg/mL AMP of SEQ ID NO: 17 was mixed with 10⁵ cells to allow a reaction for 10 min, and a resulting mixture was centrifuged at 1,000 rpm for 3 min. An experimental image was shown in FIG. 7. Experimental results show that the leukemia cells clump.

### (9) Killing of the AMP for Liver Cancer Cells

HypG2 cell line (liver cancer cells) was taken. 20 μL of a solution of 1 μg/mL AMP of SEQ ID NO: 15 was mixed with 10⁵ cells to allow a reaction for 10 min, and a resulting mixture was centrifuged at 1,000 rpm for 3 min. An experimental image was shown in FIG. 8. Experimental results show that the Liver cancer cells clump, and are broken and deformed.

### (10) Influence of the AMP on RBCs

Human RBCs were taken. 20 μL of a solution of 1 μg/mL AMP of SEQ ID NO: 27 was mixed with 2% RBCs to allow a reaction for 10 min, and a resulting mixture was centrifuged at 1,000 rpm for 3 min. An experimental image was shown in FIG. 9. Experimental results show that the human RBCs clump, and there is no obvious hemolysis.

The above are merely preferred examples of the present application, and are not intended to limit the present application. Any modification, equivalent substitution, and improvement without departing from the spirit and principle of the present application should fall within the protection scope of the present application.

1. An antimicrobial peptide or peptide derivative, comprising at least one selected from the group consisting of the following amino acid sequences: 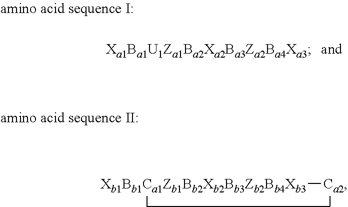
wherein Xₐ₁, Bₐ₁, U₁, Zₐ₁, Bₐ₂, Xₐ₂, Bₐ₃, Zₐ₂, Bₐ₄, Xₐ₃, X_{b1}, B_{b1}, Cₐ₁, Z_{b1}, B_{b2}, X_{b2}, B_{b3}, Z_{b2}, B_{b4}, X_{b3}, and Cₐ₂ each are independently an amino acid.

2. The antimicrobial peptide or peptide derivative according to claim 1, wherein
Xₐ₁, Xₐ₂, Xₐ₃, X_{b1}, X_{b2}, and X_{b3} each are independently at least one selected from the group consisting of positively-charged residues; and/or
Bₐ₁, Bₐ₂, Bₐ₃, Bₐ₄, B_{b1}, B_{b2}, B_{b3}, and B_{b4} each are independently at least one selected from the group consisting of hydrophobic side chain-containing residues; and/or
U₁ is one selected from the group consisting of Gly, Pro, Cys, and Cys(R), wherein (R) represents a protective group for a Cys disulfide bond; and/or
Zₐ₁, Zₐ₂, Z_{b1}, and Z_{b2} each are independently at least one selected from the group consisting of non-polar residues; and/or
Cₐ₁ and Cₐ₂ each are independently one selected from the group consisting of Cys and Cys(R).

3. The antimicrobial peptide or peptide derivative according to claim 2, wherein
Xₐ₁, Xₐ₂, Xₐ₃, X_{b1}, X_{b2}, and X_{b3} each are independently one, two, or three selected from the group consisting of Arg, His, Lys, Orn, Har, Dab, and unnatural amino acids; and/or
Bₐ₁, Bₐ₂, Bₐ₃, Bₐ₄, B_{b1}, B_{b2}, B_{b3}, and B_{b4} each are independently two, three, or four selected from the group consisting of Ala, Val, Ile, Leu, Met, and unnatural amino acids; and/or
Zₐ₁, Zₐ₂, Z_{b1}, and Z_{b2} each are independently one selected from the group consisting of Asn, Gln, Ser, Thr, and unnatural amino acids.

4. The antimicrobial peptide or peptide derivative according to claim 2, wherein the Cys disulfide bond is selected from one of the group consisting of acetamidomethyl, methyl methanethiosulfonate, and other Cys protective groups.

5. The antimicrobial peptide or peptide derivative according to claim 1, wherein the amino acid sequence I and/or the amino acid sequence II have/has at least one amino acid modified.

6. The antimicrobial peptide or peptide derivative according to claim 5, wherein the modification comprises at least one selected from the group consisting of phosphorylation, halogenation, acetylation, cyclization, and terminal capping.

7. The antimicrobial peptide or peptide derivative according to claim 1, wherein the amino acid sequence I and/or the amino acid sequence II comprise/comprises at least one motif.

8. The antimicrobial peptide or peptide derivative according to claim 1, wherein the antimicrobial peptide or peptide derivative is a multimer formed by the amino acid sequence I and/or the amino acid sequence II.

9. The antimicrobial peptide or peptide derivative according to claim 1, wherein the antimicrobial peptide or peptide derivative comprises amino acid sequences shown in SEQ ID NO: 1 to SEQ ID NO: 39.

10. The antimicrobial peptide or peptide derivative according to claim 1, wherein at least one peptide chain of the antimicrobial peptide or peptide derivative forms a nanostructure.

11. The antimicrobial peptide or peptide derivative according to claim 10, wherein the nanostructure comprises at least one selected from the group consisting of a micelle, a vesicle, a nanotube, and a nanoribbon.

12. A substitute for an antimicrobial peptide or peptide derivative, wherein the substitute is obtained by substituting at least one amino acid in the amino acid sequence I and/or the amino acid sequence II in the antimicrobial peptide or peptide derivative according to claim 1.

13. An antimicrobial peptide or peptide derivative composition, comprising:
an antimicrobial peptide or peptide derivative, wherein the antimicrobial peptide or peptide derivative comprises at least one selected from the group consisting of the following amino acid sequences: 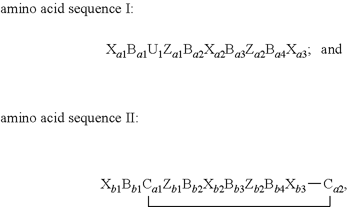
wherein Xₐ₁, Bₐ₁, U₁, Zₐ₁, Bₐ₂, Xₐ₂, Bₐ₃, Zₐ₂, Bₐ₄, Xₐ₃, X_{b1}, B_{b1}, Cₐ₁, Z_{b1}, B_{b2}, X_{b2}, B_{b3}, Z_{b2}, B_{b4}, X_{b3}, and Cₐ₂ each are independently an amino acid; and/or
a substitute, wherein the substitute is obtained by substituting at least one amino acid in the amino acid sequence I and/or the amino acid sequence II in the antimicrobial peptide or peptide derivative; and
at least one pharmaceutically acceptable carrier and/or solvent.

14. The antimicrobial peptide or peptide derivative composition according to claim 13, further comprising at least one selected from the group consisting of an excipient, an isotonic agent, an absorption retarder, and a basal polymer.

15. A preparation method of the antimicrobial peptide or peptide derivative composition according to claim 13, comprising the following step:
mixing the antimicrobial peptide or peptide derivative with the solvent.

16. An application of the antimicrobial peptide or peptide derivative composition according to claim 14 in packaging, food processing terminal, clothing, medical supplies, medical instruments, personal hygiene products, disinfectants, detergents, anti-infective drugs, anti-inflammatory drugs, and infinite cell proliferation-inhibiting drugs.

17. The antimicrobial peptide or peptide derivative according to claim 5, wherein
Xₐ₁, Xₐ₂, Xₐ₃, X_{b1}, X_{b2}, and X_{b3} each are independently at least one selected from the group consisting of positively-charged residues; and/or
Bₐ₁, Bₐ₂, Bₐ₃, Bₐ₄, B_{b1}, B_{b2}, B_{b3}, and B_{b4} each are independently at least one selected from the group consisting of hydrophobic side chain-containing residues; and/or
U₁ is one selected from the group consisting of Gly, Pro, Cys, and Cys(R), wherein (R) represents a protective group for a Cys disulfide bond; and/or
Zₐ₁, Zₐ₂, Z_{b1}, and Z_{b2} each are independently at least one selected from the group consisting of non-polar residues; and/or
Cₐ₁ and Cₐ₂ each are independently one selected from the group consisting of Cys and Cys(R).

18. The antimicrobial peptide or peptide derivative according to claim 17, wherein
Xₐ₁, Xₐ₂, Xₐ₃, X_{b1}, X_{b2}, and X_{b3} each are independently one, two, or three selected from the group consisting of Arg, His, Lys, Orn, Har, Dab, and unnatural amino acids; and/or
Bₐ₁, Bₐ₂, Bₐ₃, Bₐ₄, B_{b1}, B_{b2}, B_{b3}, and B_{b4} each are independently two, three, or four selected from the group consisting of Ala, Val, Ile, Leu, Met, and unnatural amino acids; and/or
Zₐ₁, Zₐ₂, Z_{b1}, and Z_{b2} each are independently one selected from the group consisting of Asn, Gln, Ser, Thr, and unnatural amino acids.

19. The antimicrobial peptide or peptide derivative according to claim 17, wherein the Cys disulfide bond is selected from one of the group consisting of acetamidomethyl, methyl methanethiosulfonate, and other Cys protective groups.

20. The antimicrobial peptide or peptide derivative according to claim 2, wherein the antimicrobial peptide or peptide derivative is a multimer formed by the amino acid sequence I and/or the amino acid sequence II.


SEQUENCE LISTING (39 sequences)

SEQ ID NO: 1 - protein, 17 aa, Artificial Sequence
  antimicrobial peptide, fully synthetic
HAVGNIMHIASAVLVRH

SEQ ID NO: 2 - protein, 18 aa, Artificial Sequence
  antimicrobial peptide, fully synthetic
HKAVGQIMHIASAVLVRH

SEQ ID NO: 3 - protein, 19 aa, Artificial Sequence
  antimicrobial peptide, fully synthetic
HRAMPNMLKIMASLVLAKR

SEQ ID NO: 4 - protein, 19 aa, Artificial Sequence
  antimicrobial peptide, fully synthetic
HAMPNMLRIMASLVLARHK

SEQ ID NO: 5 - protein, 18 aa, Artificial Sequence
  antimicrobial peptide, fully synthetic
HKAVPNMLHIASAVLVRH

SEQ ID NO: 6 - protein, 18 aa, Artificial Sequence
  antimicrobial peptide, fully synthetic
HKAVGNMLKIASAVLVHK

SEQ ID NO: 7 - protein, 18 aa, Artificial Sequence
  antimicrobial peptide, fully synthetic
HAMPNMLRIMASAVLVKR

SEQ ID NO: 8 - protein, 18 aa, Artificial Sequence
  antimicrobial peptide, fully synthetic
HAMGNMLKIMASAVLVRH

SEQ ID NO: 9 - protein, 20 aa, Artificial Sequence
  antimicrobial peptide, fully synthetic
HRAMGQIVKMIATLVLARKH

SEQ ID NO: 10 - protein, 21 aa, Artificial Sequence
  antimicrobial peptide, fully synthetic
HKRAMPSIVKMIATLVLARKH

SEQ ID NO: 11 - protein, 19 aa, Artificial Sequence
  antimicrobial peptide, fully synthetic
RKAMPNMLRIMASAVLVHK

SEQ ID NO: 12 - protein, 20 aa, Artificial Sequence
  antimicrobial peptide, fully synthetic
RHAMGNMLHIMASLVLARKH

SEQ ID NO: 13 - protein, 20 aa, Artificial Sequence
  antimicrobial peptide, fully synthetic
RHAVPNMLRIMASLVLAHKR

SEQ ID NO: 14 - protein, 18 aa, Artificial Sequence
  antimicrobial peptide, fully synthetic
RAMPNMLKIMASAVLVHK

SEQ ID NO: 15 - protein, 19 aa, Artificial Sequence
  antimicrobial peptide, fully synthetic
RAMPNMLRIMASLVLARKH

SEQ ID NO: 16 - protein, 20 aa, Artificial Sequence
  antimicrobial peptide, fully synthetic
RKAVPNMLRIMASAVLVHKR

SEQ ID NO: 17 - protein, 19 aa, Artificial Sequence
  antimicrobial peptide, fully synthetic
RHAMGQMLRIMASLVLARK

SEQ ID NO: 18 - protein, 21 aa, Artificial Sequence
  antimicrobial peptide, fully synthetic
RKHAMPSIVKMIATLVLARKH

SEQ ID NO: 19 - protein, 20 aa, Artificial Sequence
  antimicrobial peptide, fully synthetic
RKHAMGQIVKMIATLVLAHR

SEQ ID NO: 20 - protein, 20 aa, Artificial Sequence
  antimicrobial peptide, fully synthetic
RHAVPNMLRIMASAVLVKHR

SEQ ID NO: 21 - protein, 18 aa, Artificial Sequence
  antimicrobial peptide, fully synthetic
KAVPNIMHIMASAVLVRH

SEQ ID NO: 22 - protein, 19 aa, Artificial Sequence
  antimicrobial peptide, fully synthetic
KRAMPNIVRIMASAVLVRH

SEQ ID NO: 23 - protein, 19 aa, Artificial Sequence
  antimicrobial peptide, fully synthetic
KHAMPNMLRIMASLVLARH

SEQ ID NO: 24 - protein, 19 aa, Artificial Sequence
  antimicrobial peptide, fully synthetic
KAVPNMLRIMASLVLARKH

SEQ ID NO: 25 - protein, 20 aa, Artificial Sequence
  antimicrobial peptide, fully synthetic
KHAVGQIVKMIATLVLAKHR

SEQ ID NO: 26 - protein, 20 aa, Artificial Sequence
  antimicrobial peptide, fully synthetic
KRAMPSIVKMIATAVLVRKH

SEQ ID NO: 27 - protein, 18 aa, Artificial Sequence
  antimicrobial peptide, fully synthetic
KAVPNMLKIMASLVLARH

SEQ ID NO: 28 - protein, 20 aa, Artificial Sequence
  antimicrobial peptide, fully synthetic
KHAVPNMLRIMASLVLAHKR

SEQ ID NO: 29 - protein, 20 aa, Artificial Sequence
  antimicrobial peptide, fully synthetic
KHRAVGNMLKIASLVLARHK

SEQ ID NO: 30 - protein, 19 aa, Artificial Sequence
  antimicrobial peptide, fully synthetic
KAMPNIVRIMASLVLARHK

SEQ ID NO: 31 - protein, 21 aa, Artificial Sequence
  antimicrobial peptide, fully synthetic
HRAMCPNMLKIMASLVLAKRC

SEQ ID NO: 32 - protein, 21 aa, Artificial Sequence
  antimicrobial peptide, fully synthetic
HAMCPNMLRIMASLVLARHKC

SEQ ID NO: 33 - protein, 23 aa, Artificial Sequence
  antimicrobial peptide, fully synthetic
HKRAMCPSIVKMIATLVLARKHC

SEQ ID NO: 34 - protein, 21 aa, Artificial Sequence
  antimicrobial peptide, fully synthetic
RKAMCPNMLRIMASAVLVHKC

SEQ ID NO: 35 - protein, 21 aa, Artificial Sequence
  antimicrobial peptide, fully synthetic
RAMCPNMLRIMASLVLARKHC

SEQ ID NO: 36 - protein, 21 aa, Artificial Sequence
  antimicrobial peptide, fully synthetic
RHAMCGQMLRIMASLVLARKC

SEQ ID NO: 37 - protein, 21 aa, Artificial Sequence
  antimicrobial peptide, fully synthetic
KRAMCPNIVRIMASAVLVRHC

SEQ ID NO: 38 - protein, 21 aa, Artificial Sequence
  antimicrobial peptide, fully synthetic
KHAMCPNMLRIMASLVLARHC

SEQ ID NO: 39 - protein, 22 aa, Artificial Sequence
  antimicrobial peptide, fully synthetic
KHRAVCGNMLKIASLVLARHKC